(19) United States
(12) Patent Application Publication
(10) Pub. No.: US 2008/0255651 A1
(43) Pub. Date: Oct. 16, 2008

(54) Telescoping Stability Sheath and Method of Use

(75) Inventor: Joshua Dwork, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/734,641

(22) Filed: Apr. 12, 2007

(51) Int. Cl. A61F 2/06

(52) U.S. Cl. 623/1.11

(57) ABSTRACT

A telescoping stability sheath and method of use, including a telescoping stability sheath for use with a stent delivery system having a delivery catheter and an introducer, the delivery catheter having a retractable sheath and a stability sheath stop axially slidable on the retractable sheath, the introducer having an introducer fitting slidably tightenable about the retractable sheath axially slidable within a sheath stop. The telescoping stability sheath includes a first segment slidably disposed about the retractable sheath, a second segment slidably disposed about the first segment, and a stability fitting attached to at least one of the first segment and the second segment. The stability fitting fixes a set length between the introducer fitting and the stability sheath stop when the stability fitting is engaged to lock the first segment to the second segment.

### TECHNICAL FIELD

The technical field of this disclosure is medical implantation devices, particularly, a telescoping stability sheath for use with a stent delivery system.

### BACKGROUND OF THE INVENTION

Wide ranges of medical treatments have been developed using endoluminal prostheses, which are medical devices adapted for temporary or permanent implantation within a body lumen, such as naturally occurring or artificially made lumens. Examples of lumens in which endoluminal prostheses may be implanted include arteries such as those located within coronary, mesentery, peripheral, or cerebral vasculature; arteries; gastrointestinal tract; biliary tract; urethra; trachea; hepatic shunts; and fallopian tubes. Various types of endoluminal prostheses have also been developed with particular structure to modify the mechanics of the targeted luminal wall.

A number of vascular devices have been developed for replacing, supplementing, or excluding portions of blood vessels. These vascular devices include endoluminal vascular prostheses, such as stents, and stent grafts, which provide a graft material to direct flow and reduce pressure on the vascular wall. The tubular endoluminal prosthesis is introduced in a small diameter condition and expands or is expanded at the deployment site. The tubular endoluminal prosthesis can be self-expanding so that the tubular endoluminal prosthesis expands in diameter once restraints holding it in the small diameter are removed.

One of the many uses for stents is to treat peripheral vascular disease, which affects more than 12 million people in the U.S., or one in 15 adults. Narrowing of vessels and blockages restrict blood circulation, mainly in arteries leading to the kidneys, stomach, arms, legs and feet. For peripheral vascular disease of the legs, blockages in the iliac artery can be removed and the patency of the artery maintained with a stent.

As explained in detail in U.S. Pat. No. 5,906,619, discussing FIGS. 6 and 7 therein, the full disclosure of which is incorporated herein by reference: Introducer sheaths generally provide hemostasis around catheters, guidewires, other invasive surgical implements of various sizes and configurations. Such introducer sheaths typically include a resilient sealing body which radially engages the outermost layer of the delivery system. As it is generally desirably to leave the internal prosthesis at a fixed position while withdrawing the sheath proximally, therefore friction between introducer valve and sheath is generally disadvantageous. However, by coupling an outer tube (introducer engaging tube and/or a portion of the stability sheath) to a housing (formed by part of a stability a stability sheath—where the outer tube and housing collectively form the stability sheath), and by providing an actuation mechanism which withdraws the sheath relative to a middle shaft and housing, friction between the outer tube and introducer valve may be used to help restrain the prosthesis at the target location during deployment.

To facilitate insertion of outer tube into the introducer valve, a distal end of tubular body may be tapered. In some embodiments, introducer valve may be actuated once outer tube and the prosthesis are positioned, compressing the sealing body against the outer tube to lock the prosthesis in place. A particularly advantageous actuatable introducer valve is described in U.S. Pat. No. 6,276,661, the full disclosure of which is incorporated herein by reference.

An alternative system and method for maintaining the position of the prosthesis within patient body is illustrated in FIG. 7 of U.S. Pat. No. 5,906,619. In this embodiment, the actuation mechanism for withdrawing sheath relative to shaft is contained in a removable actuation housing. The housing is coupled to introducer valve using a brace rod.

Transitioning to a discussion of elements related to the present embodiments, FIG. 1 is a cross section view of the several pieces of a stent delivery system. The stent delivery system 50, such as the Complete SE stent delivery system manufactured by Medtronic, Inc., of Minneapolis, Minn., includes a delivery catheter 48 and an introducer 62. The delivery catheter 48 includes a stability sheath 52 disposed about a retractable sheath 54, which is disposed about an inner shaft 56. The handle 58 at the proximal end of the delivery catheter 48 is attached to the stability sheath 52 and the inner shaft 56. The retractable sheath 54 is attached to a button 60, which extends through an opening 61 in the handle 58 and allows the retractable sheath 54 to slide axially with respect the stability sheath 52 and the inner shaft 56. The introducer 62 has an introducer fitting 64 with a tightenable seal 65, such as an O-ring seal, at the proximal end.

In operation, the introducer 62 is inserted in a artery in the patient with the distal end of the introducer 62 terminating short of the proximal end of the deployment site and the introducer fitting 64 remaining outside of the patient. The delivery catheter 48 includes a self-expanding stent 66: the self-expanding stent 66 is located adjacent to a distal end portion of the inner shaft 56 which prevents the self-expanding stent 66 from moving axially toward the handle 58 when deployed and is restrained (constrained) to a small diameter by the retractable sheath 54. The distal end of the delivery catheter 48 is advanced through the introducer 62 into the vasculature of the patient until the self-expanding stent 66 is located at the deployment site. The introducer fitting 64 of the introducer 62 is tightened onto the stability sheath 52 to fix the introducer fitting 64 to the stability sheath 52, locate the distal end of the delivery catheter 48 at the deployment site, and provide a fluid seal. To initiate stent deployment the button 60 is moved axially toward the proximal end of handle 58 which thereby retracts the retractable sheath 54. The inner shaft 56 holds the self-expanding stent 66 axially at the deployment site. The self-expanding stent 66 expands as the distal end of the retractable sheath 54 moves toward the handle 58 and uncovers and frees the self-expanding stent 66. The stability sheath 52 defines the path and the distance for the retractable sheath 54 to follow, avoiding problems with direct friction between the introducer fitting 64 and the retractable sheath 54. The stability sheath acts as a distance stabilizing member to help set and maintain the distance between the handle and the introducer positioned in the patient's artery to thereby promotes accurate deployment of the stent at the deployment site. The stability sheath 52 also reduces or prevents the problem of the movement of the distal end of the retractable sheath 54 from the deployment site which may occur when the stability sheath 52 is omitted.

The present stent delivery system presents certain opportunities for improvement. The inner diameter of the introducer is sized to fit over the stability sheath, so the potential reduction in the diameter of the introducer is limited by the size of the outside diameter of the stability sheath, whose reduction in diameter is limited by the outside diameter of the retractable sheath whose inside diameter at the stent holding location must be large enough to fit over the stent. For example, if the retractable sheath of a stent delivery system is 5 Fr, the addition of a stability sheath could make the system a 6 Fr system which forces the user to use an introducer known as a 6 Fr introducer nominally sized to receive a 6 Fr device. Requiring the use of a larger diameter introducer reduces possible applications of the stent delivery system. A larger introducer is less flexible, making it more difficult to maneuver through the vasculature to reach remote deployment sites. A larger introducer is also too large to fit into narrow arteries.

It would be desirable to have stability sheath and method of use that would overcome the above disadvantages.

### SUMMARY OF THE INVENTION

One aspect according to the present invention provides a telescoping stability sheath for use with any stent delivery system having a delivery catheter and an introducer, i.e., one without a stability sheath. The delivery catheter has a retractable stability sheath stop near the handle of the delivery system, and a stability sheath stop axially slidable on the retractable sheath, the introducer having an introducer fitting slidable and tightenable about the retractable sheath. The telescoping stability sheath includes a first segment slidably disposed about the retractable sheath, a second segment slidably disposed about the first segment, and a stability fitting attached to at least one of the first segment and the second segment. The stability fitting fixes a set length between the introducer fitting and the stability sheath stop when the stability fitting is engaged to lock the first segment to the second segment.

Another aspect according to the present invention provides a system for delivering a stent into a body lumen, the system including a delivery catheter, the delivery catheter having a retractable sheath and a stability sheath stop near the handle of the delivery system, an introducer, the introducer having an introducer fitting slidably tightenable about the retractable sheath; and a telescoping stability sheath. The telescoping stability sheath includes a first segment slidably disposed about the retractable sheath; a second segment slidably disposed about the first segment; and a stability fitting attached to at least one of the first segment and the second segment. The stability fitting fixes a set length between the introducer fitting and the stability sheath stop when the stability fitting is engaged to lock the first segment to the second segment.

Another aspect according to the present invention provides a method of delivering a stent into a body lumen, the method including providing an introducer having an introducer first end and an introducer second end; inserting the introducer first end into the body lumen; providing a delivery catheter having a stability sheath stop; inserting the delivery catheter into the introducer with a set length of the delivery catheter between the introducer second end and the stability sheath stop; and fixing the set length between the introducer second end and the stability sheath stop.

Another aspect according to the present invention provides a system for delivering a stent into a body lumen, the system including an introducer having an introducer first end and an introducer second end; a delivery catheter a retractable sheath and a stability sheath stop axially slidable on the retractable sheath; and means for fixing a set length between the introducer second end and the stability sheath stop when the introducer first end is in the body lumen, the retractable sheath is in the introducer, and the set length is an axial distance between the introducer second end and the stability sheath stop.

The foregoing and other features and advantages will become further apparent from the following detailed description, read in conjunction with the accompanying drawings. The detailed description and drawings are merely illustrative.

### BRIEF DESCRIPTION OF THE DRAWINGS

FIG. 1 is a cross section view of a prior art stent delivery system;

FIGS. 2A-2D are side views of one embodiment of a telescoping stability sheath in accordance with the present invention;

FIGS. 3A-3D are side views of another embodiment of a telescoping stability sheath in accordance with the present invention; and

FIG. 4 is a flowchart of a method of delivering a stent into a body lumen with a telescoping stability sheath in accordance with the present invention.

### DETAILED DESCRIPTION

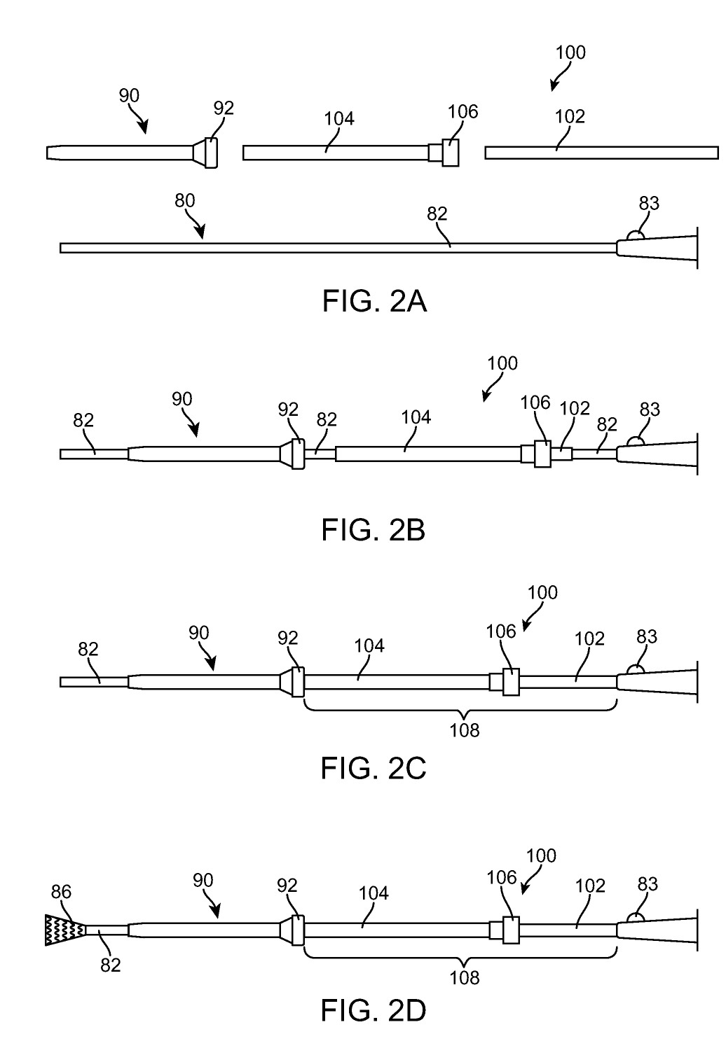

FIGS. 2A-2D, in which like elements share like reference numbers, are side views of one embodiment of a telescoping stability sheath. FIGS. 2A, 2B, 2C, and 2D illustrate the telescoping stability sheath in an exploded, collapsed, extended, and deployment condition, respectively. In this embodiment, the telescoping stability sheath is not fixed to the delivery catheter.

The telescoping stability sheath 100 can be used with a stent delivery system including a delivery catheter 80 and an introducer 90. The delivery catheter 80 includes a retractable sheath 82 and a stability sheath stop 83, such as a handle, strain relief portion, or the like, at the proximal end of the retractable sheath 82. As defined herein, the stability sheath stop 83 is any portion or feature of the delivery catheter 80 that is fixed to or can engage the proximal end of the telescoping stability sheath and prevent the proximal end of the telescoping stability sheath from sliding axially past the stability sheath stop 83. The stability sheath stop 83 is not attached to the retractable sheath 82, so that the retractable sheath 82 can be withdrawn axially to deploy a stent 86. The retractable sheath 82 can be connected to an actuator (not shown), such as a button, slider, handle, or other mechanism operable to move the retractable sheath 82 relative to the stability sheath stop 83. The delivery catheter 80 also includes an inner shaft (not shown) fixed to the stability sheath stop 83 and holding the stent 86 at the distal end of the retractable sheath 82. The introducer 90 includes an introducer fitting 92 selectively slidable about the retractable sheath 82. The introducer fitting 92 can include seals, such as O-rings, in contact with the retractable sheath 82. The introducer fitting 92 is tightenable to provide a fluid seal about the retractable sheath 82 to reduce blood loss from the patient during stent delivery, while allowing the introducer 90 to slide relative to the retractable sheath 82. In one embodiment, the stent delivery system also includes a guidewire (not shown) slidable within the delivery catheter 80. The guidewire can be threaded through a guide catheter threaded through the introducer, the guide catheter is then removed leaving the guidewire in place in the body lumen of the patient and the delivery catheter 80 introduced over the guidewire.

As used herein, proximal and distal are defined in relation to the operator performing the stent delivery procedure, so that proximal end is near the operator and distal end is away from the operator. For example, the proximal end of the delivery catheter is the end from which the operator manipulates the delivery catheter and the distal end of the delivery catheter is the end from which the stent is deployed inside the patient.

The telescoping stability sheath 100 is disposed about the retractable sheath 82 of the delivery catheter 80 between the introducer 90 and the stability sheath stop 83. In one embodiment, the telescoping stability sheath 100 contacts the introducer fitting 92 of the introducer 90. The telescoping stability sheath 100 includes a first segment 102 disposed about the retractable sheath 82, a second segment 104 slidably disposed about the first segment 102, and a stability fitting 106 attached to at least one of the first segment 102 and the second segment 104. In this example, the stability fitting 106 is attached to the second segment 104.

When engaged, the stability fitting 106 locks the first segment 102 and the second segment 104 into a set length 108 between the introducer 90 and the stability sheath stop 83. A set length 108 between the introducer 90 and the stability sheath stop 83 allows the retractable sheath 82 to be retracted into the handle to deploy a stent at the distal end of the retractable sheath 82. The distance between the stability sheath stop 83 and the distal end of the introducer 90 is kept constant because the telescoping stability sheath 100 maintains the distance between the stability sheath stop 83 and the proximal end of the introducer 90. The retractable sheath 82 slides within the introducer fitting 92, which maintains a fluid seal.

The first segment 102 and the second segment 104 can be made of any material sufficiently incompressible to maintain the set length 108 between the stability sheath stop 83 and the proximal end of the introducer 90. In one embodiment, the segments 102, 104 are made of extruded plastic, such as fluoropolymer or the like. In one embodiment, the segments 102, 104 are laterally flexible to follow the bending shape of the retractable sheath 82. In one embodiment, the segments 102, 104 are reinforced with fiber, wire, or the like to increase strength and/or oriented flexibility and resistance to kinking while remaining almost incompressible axially. Those skilled in the art will appreciate that the first segment 102 and the second segment 104 can be the same or different depending on the particular application. The telescoping stability sheath 100 can be mounted on the retractable sheath 82 so that the first segment 102 can be near the stability sheath stop 83 or near the introducer 90 as desired, before being extended for use.

The segments 102, 104 can be sized to fit the delivery catheter 80. The first segment 102 has a smaller diameter than and slides inside of the second segment 104. The segments 102, 104 can be any diameter sufficient to fit over the retractable sheath 82 while being restrained by the stability sheath stop 83 and the introducer 90. One diameter of telescoping stability sheath 100 can be used with a number of delivery catheters having a smaller diameter of retractable sheath 82, e.g., a 5 French telescoping stability sheath 100 can be used with delivery catheters 80 having up to a 4 French retractable sheath 82. The length of the segments 102, 104 can be any length sufficient to reach between the stability sheath stop 83 and the proximal end of the introducer 90, with sufficient overlap to allow the stability fitting 106 to lock the segments 102, 104 to the set length 108. In one embodiment, the segments 102, 104 are slightly longer one half the length of the set length 108.

The telescoping stability sheath can be made of more than the first segment 102 and the second segment 104 as desired. In one example, the second segment 104 can include a first sub-segment slidably disposed about the first segment 102, a second sub-segment slidably disposed about the first sub-segment, and a second stability fitting attached to at least one of the first sub-segment and the second sub-segment. The second stability fitting locks the first segment and the second segment to a second set length when engaged. Those skilled in the art will appreciate that the set length 108 between the stability sheath stop 83 and the proximal end of the introducer 90 can be filled with as many segments as desired for a particular application, with a stability fitting between each adjacent pair of segments.

The stability fitting 106 is attached to at least one of the first segment 102 and the second segment 104. The stability fitting 106 can be any fitting on the first segment 102 and the second segment 104 for locking the combination of the first segment 102 and the second segment 104 to maintain a set length 108. In one embodiment, the stability fitting 106 is a compression fitting, such as a Touhy Borst fitting, attached to the second segment 104 to clamp onto the first segment 102.

The first segment 102 and the second segment 104 can be attached to the introducer 90 and/or stability sheath stop 83. The attachment can be permanent, or can be separable, with a complementary fitting operable to make the connection between the segment and the introducer 90 or stability sheath stop 83. In one embodiment, the distal end of one of the segments 102, 104 is attached to the introducer 90. In one embodiment, the proximal end of one of the segments 102, 104 is attached to the stability sheath stop 83. In one embodiment, the distal end of one of the segments 102, 104 is tapered to engage the introducer fitting.

Referring to FIG. 2D, the telescoping stability sheath 100 is shown in the deployment condition in which the stent 86 is being deployed. The actuator (not shown) is moved proximally relative to the stability sheath stop 83, retracting the retractable sheath 82 from over the stent 86. The stent 86 is self-expanding and expands into the lumen (not shown). The introducer 90 and the stability sheath stop 83 remain a set length apart because the extended telescoping stability sheath 100 maintains the set length 108. Therefore, the distal end of the introducer 90 does not move axially relative to the stent 86 as the retractable sheath 82 retracts. The retractable sheath slides relative to the introducer 90 with the introducer fitting 92 maintaining the fluid seal.

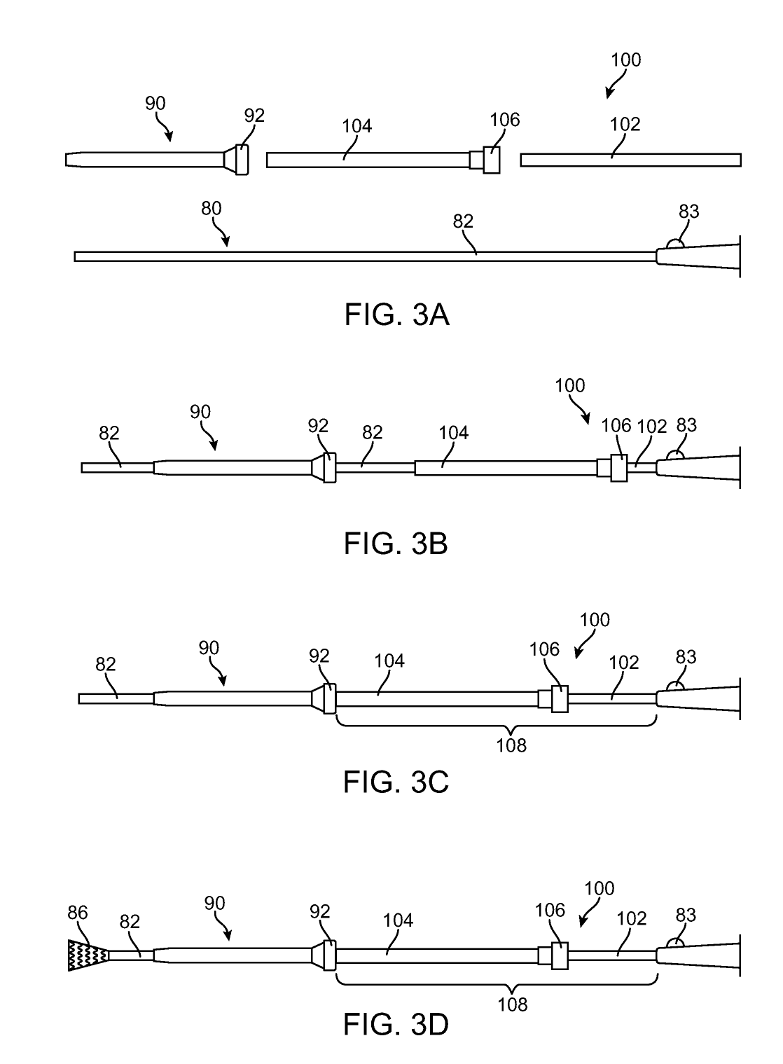

FIGS. 3A-3D, in which like elements share like reference numbers with each other and with FIGS. 3A-3D, are side views of another embodiment of a telescoping stability sheath made in accordance with the present invention. FIGS. 3A, 3B, 3C, and 3D illustrate the telescoping stability sheath in an exploded, collapsed, extended, and deployment condition, respectively. In this embodiment, one of the segments of the telescoping stability sheath is attached to the delivery catheter.

In this example, the first segment 102 is attached to the stability sheath stop 83 of the delivery catheter 80, so that the delivery catheter 80 is attached to the telescoping stability sheath 100. The second segment 104 is adjusted axially when the distal end of the retractable sheath 82 is at the deployment site so that the distal end of the second segment 104 contacts the proximal end of the introducer 90. The stability fitting 106 is locked to provide and maintain a set length 108 between the introducer 90 and the stability sheath stop 83.

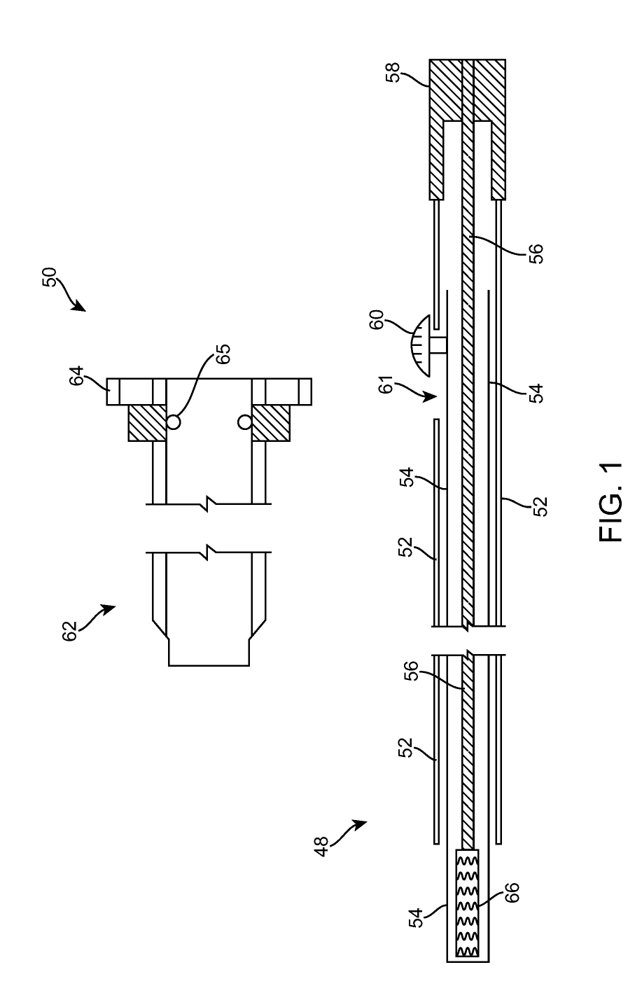

Those skilled in the art will appreciate that the second segment 104 with the stability fitting 106 can be used with the stent delivery system of FIG. 1 in certain applications. Referring to FIG. 1, when the stability sheath 52 disposed about the retractable sheath 54 is short such that the stability sheath 52 does not reach to the introducer 62 when the introducer 62 is inserted in an artery in the patient, a second segment 104 of FIGS. 3A-3D can be used to lock on the stability sheath 52 to maintain the set length between the introducer 62 and the handle 58, which acts as the sheath stop. Such an application can occur in procedures involving the iliac artery entered from the femoral artery on the same side when using a delivery catheter with a short stability sheath.

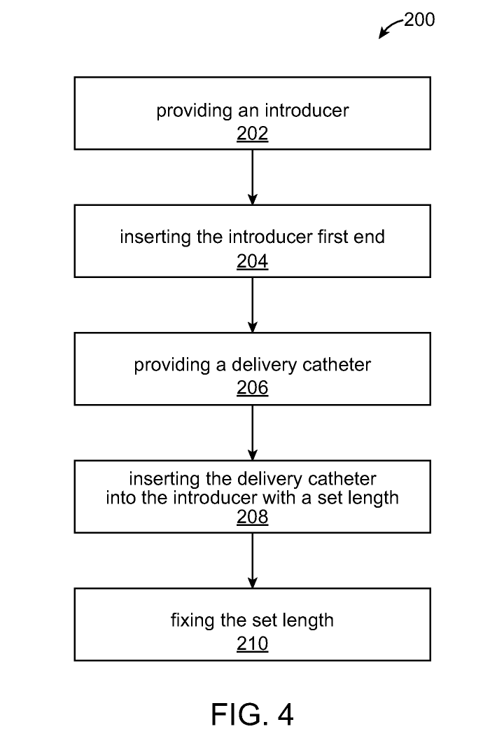

FIG. 4 is a flowchart of a method of delivering a stent into a body lumen with a telescoping stability sheath made in accordance with the present invention. The method 200 includes providing an introducer 202, inserting the introducer first end 204, providing a delivery catheter 206, inserting the delivery catheter into the introducer with a set length 208, and fixing the set length 210.

The providing an introducer 202 includes providing an introducer having an introducer first end and an introducer second end. In one embodiment, the introducer has an introducer fitting at or near the introducer second end to provide a slidable seal about the retractable sheath of the delivery catheter. An exemplary introducer is illustrated in FIGS. 2-3.

Referring to FIG. 4, the inserting the introducer first end 204 includes inserting the introducer first end into the body lumen. The introducer first end is advanced distally into the body lumen until the introducer first end is located just proximally of the deployment site.

The providing a delivery catheter 206 includes providing a delivery catheter having a sheath stop. The stability sheath stop is located at or near the proximal end of the delivery catheter. In one embodiment, the delivery catheter has a retractable sheath axially slidable within the sheath stop. An exemplary delivery catheter is illustrated in FIGS. 2-3.

Referring to FIG. 4, the step of inserting the delivery catheter into the introducer with a set length 208 includes inserting the delivery catheter into the introducer with a set length of the delivery catheter between the introducer second end and the sheath stop. The set length of the delivery catheter is the axial length along the delivery sheath between the introducer second end that remains out of the patient and the sheath stop at the proximal end of the delivery sheath. In one embodiment, the inserting the delivery catheter 208 also includes tightening the introducer fitting of the introducer onto the retractable sheath of the delivery catheter to provide a seal and prevent leakage from the body lumen through the introducer. The retractable sheath is slidable within the tightened introducer fitting so the retractable sheath can be withdrawn to deploy a stent at the deployment site. In one embodiment, the inserting the delivery catheter 208 also includes positioning a guidewire through the introducer into the body lumen and inserting the delivery catheter through the introducer over the guidewire. In one embodiment, the inserting the delivery catheter 208 also includes adjusting a telescoping stability sheath about the retractable sheath of the delivery catheter. The telescoping stability sheath can include a first segment, a second segment slidably disposed about the first segment, and a stability fitting attached to at least one of the first segment and the second segment. An exemplary telescoping stability sheath is illustrated in FIGS. 2-3.

Referring to FIG. 4, the fixing the set length 210 includes fixing the set length between the introducer second end and the sheath stop. In one embodiment, the fixing the set length 210 includes locking the telescoping stability sheath to the set length between the introducer second end and the sheath stop to maintain the relative axial position of the introducer and the sheath stop. When the telescoping stability sheath has a stability fitting with a first segment and a second segment, the locking the telescoping stability sheath can include engaging the stability fitting to lock the first segment to the second segment.

The method 200 can continue with deployment of a stent at the deployment site. The retractable sheath of the delivery catheter is axially slidable within the sheath stop and extends beyond the introducer first end into the body lumen. The distal end of the retractable sheath restrains a stent at the deployment site. The retractable sheath is withdrawn, such as by pulling the proximal end of retractable sheath through the sheath stop, to release the stent at the deployment site.

While specific embodiments of the invention are disclosed herein, various changes and modifications can be made without departing from the spirit and scope of the invention.

1. A telescoping stability sheath for use with a stent delivery system having a delivery catheter and an introducer, the delivery catheter having a retractable sheath and a retractable sheath axially slidable within a stability sheath stop, the introducer having an introducer fitting slidably tightenable about the retractable sheath, the telescoping stability sheath comprising:
a first segment slidably disposed about the retractable sheath;
a second segment slidably disposed about the first segment; and
a stability fitting attached to at least one of the first segment and the second segment;
wherein the stability fitting fixes a set length between the introducer fitting and the stability sheath stop when the stability fitting is engaged to lock the first segment to the second segment.

2. The telescoping stability sheath of claim 1 wherein the stability fitting is a compression fitting attached to the second segment.

3. The telescoping stability sheath of claim 2 wherein the stability fitting is a Touhy Borst fitting.

4. The telescoping stability sheath of claim 1 wherein the second segment comprises:
a first sub-segment slidably disposed about the first segment;
a second sub-segment slidably disposed about the first sub-segment; and
a second stability fitting attached to at least one of the first sub-segment and the second sub-segment, the second stability fitting fixing the first sub-segment and the second sub-segment to a second set length when engaged.

5. The telescoping stability sheath of claim 1 wherein a proximal end of one of the first segment and the second segment is attached to the stability sheath stop.

6. The telescoping stability sheath of claim 1 wherein a distal end of one of the first segment and the second segment is tapered to engage the introducer fitting.

7. The telescoping stability sheath of claim 1 further comprising a complementary fitting operable to connect a distal end of one of the first segment and the second segment to the introducer fitting when engaged.

8. The telescoping stability sheath of claim 1 further comprising a complementary fitting operable to connect a proximal end of one of the first segment and the second segment to the stability sheath stop when engaged.

9. A system for delivering a stent into a body lumen, the system comprising:
a delivery catheter, the delivery catheter having a retractable sheath and a stability sheath stop through which the retractable sheath can slide;
an introducer, the introducer having an introducer fitting slidably tightenable about the retractable sheath; and
a telescoping stability sheath comprising:
a first segment slidably disposed about the retractable sheath;
a second segment slidably disposed about the first segment; and
a stability fitting attached to at least one of the first segment and the second segment;
wherein the stability fitting fixes a set length between the introducer fitting and the stability sheath stop when the stability fitting is engaged to lock the first segment to the second segment.

10. The system of claim 9 wherein the stability fitting is a compression fitting attached to the second segment.

11. The system of claim 10 wherein the stability fitting is a Touhy Borst fitting.

12. The system of claim 9 wherein the second segment comprises:
a first sub-segment slidably disposed about the first segment;
a second sub-segment slidably disposed about the first sub-segment; and
a second stability fitting attached to at least one of the first sub-segment and the second sub-segment, the second stability fitting fixing the first sub-segment and the second sub-segment to a second set length when engaged.

13. The system of claim 9 wherein a proximal end of one of the first segment and the second segment is attached to the stability sheath stop.

14. The system of claim 9 wherein a distal end of one of the first segment and the second segment is tapered to engage the introducer fitting.

15. The system of claim 9 further comprising a complementary fitting operable to connect a distal end of one of the first segment and the second segment to the introducer fitting when engaged.

16. The system of claim 9 further comprising a complementary fitting operable to connect a proximal end of one of the first segment and the second segment to the stability sheath stop when engaged.

17. The system of claim 9 further comprising a guidewire slidable within the delivery catheter.

18. A method of delivering a stent into a body lumen, the method comprising:
providing an introducer having an introducer first end and an introducer second end;
inserting the introducer first end into the body lumen;
providing a delivery catheter having a stability sheath stop;
inserting the delivery catheter into the introducer with a set length of the delivery catheter between the introducer second end and the stability sheath stop; and
fixing the set length between the introducer second end and the stability sheath stop.

19. The method of claim 18 wherein the introducer has an introducer fitting at the introducer second end, the delivery catheter has a retractable sheath axially slidable within the stability sheath stop, and the inserting the delivery catheter further comprises tightening the introducer fitting onto the retractable sheath.

20. The method of claim 18 wherein the inserting the delivery catheter comprises inserting a guidewire through the introducer into the body lumen and inserting the delivery catheter through the introducer over the guidewire.

21. The method of claim 18 wherein the delivery catheter has a retractable sheath axially slidable within the stability sheath stop, the inserting the delivery catheter further comprises positioning a telescoping stability sheath about the retractable sheath, and the fixing the set length comprises locking the telescoping stability sheath to the set length between the introducer second end and the stability sheath stop.

22. The method of claim 21 wherein the telescoping stability sheath comprises a first segment, a second segment slidably disposed about the first segment, and a stability fitting attached to at least one of the first segment and the second segment, and the locking comprises engaging the stability fitting to lock the first segment to the second segment.

23. The method of claim 18 wherein the delivery catheter has a retractable sheath axially slidable within the stability sheath stop, the retractable sheath extending beyond the introducer first end and restraining a stent at a deployment site in the body lumen, and further comprising withdrawing the retractable sheath to release the stent at the deployment site.

24. A system for delivering a stent into a body lumen, the system comprising:
an introducer having an introducer first end and an introducer second end;
a delivery catheter a retractable sheath and a stability sheath stop axially slidable on the retractable sheath; and
means for fixing a set length between the introducer second end and the stability sheath stop when the introducer first end is in the body lumen, the retractable sheath is in the introducer, and the set length is an axial distance between the introducer second end and the stability sheath stop.

25. The system of claim 24 wherein the introducer includes means for slidably sealing about the retractable sheath.

26. The system of claim 24 wherein the fixing means comprises a first segment, a second segment slidably disposed about the first segment, and means for locking the first segment to the second segment.
